(19) United States
(12) Patent Application Publication
(10) Pub. No.: US 2011/0275874 A1
(43) Pub. Date: Nov. 10, 2011

(54) REACTOR AND METHOD FOR PROPYLENE PRODUCTION BY STRATIFIED INJECTION OF HEAVY OIL AND LIGHT OLEFINS

(75) Inventors: Chunyi LI, Qingdao (CN); Chaohe Yang, Qingdao (CN); Yongqing Hu, Qingdao (CN); Yibin Liu, Qingdao (CN); Xiaobo Chen, Qingdao (CN); Qiang Zhang, Qingdao (CN); Honghong Shan, Qingdao (CN)

(73) Assignee: China University of Petroleum (East China), Qingdao (CN)

(21) Appl. No.: 12/952,924

(22) Filed: Nov. 23, 2010

(30) Foreign Application Priority Data
May 10, 2010 (CN) 201010166878.4

(51) Int. Cl. C07C 4/06; B01J 19/00

(52) U.S. Cl. 585/653; 422/129

(57) ABSTRACT

A riser reactor for propylene production, comprises pipelines with an upper part and a lower part, the upper part is a straight pipeline, and the lower part is a expanding-diameter pipeline, and the expanding-diameter pipeline is a circular truncated cone-shaped pipeline with an angle of 1˜60 o between generatrix and axes, wherein, the diameter of upper surface of the circular truncated cone-shaped pipeline is larger than or equal to the diameter of the straight pipeline. Employed the riser reactor, propylene is produced by the method of catalytic cracking with stratified injections of heavy oil and light olefins. Average gas linear velocity in axial direction of the straight pipeline of riser reactor is 3˜25 m/s, and that in the expanding-diameter pipeline is 0.1 to 5 m/s. The outlet temperature of the riser reactor is 460 to 600° C., and the light olefins are no more than 50% of the total feedstock by weight. The yield and selectivity of propylene was improved, accompanied with production of diesel and gasoline with high octane number.

### FIELD OF THE INVENTION

The invention relates to a method for production of propylene. To be specific, it relates to a method for propylene production with consideration to diesel and gasoline with high octane number by catalytic cracking with stratified injection of heavy oil and light olefins.

### BACKGROUND OF THE INVENTION

Propylene, one of the most important basic materials in chemical industry, can be produced by steam cracking of naphtha, catalytic cracking of heavy oil, dehydrogenation of propane and metathesis with ethylene and 2-butylene, etc. From the aspect of capacity of propylene production, steam cracking of naphtha is of the greatest contribution, followed by catalytic cracking of heavy oil. The latter two processes contributed a little for propylene output.

There are several disadvantages of naphtha steam cracking process for propylene production: shortage of qualified naphtha feedstock, high energy consumption, low ratio of propylene-to-ethylene and low yield of high added value products. However, catalytic cracking of heavy oil process made great development in recent years due to its advantages, consisting of abundance in feedstock, mild reaction conditions, low energy consumption, high yield and selectivity of propylene, and high yield of high value-added products.

Several technologies of heavy oil catalytic cracking for propylene production had been developed, such as: DCC-I, DCC-II, Petroriser and Milos. The TMP technology, developed by China university of Petroleum (East China), can not only produce propylene with high yield and selectivity using residual oil as feedstock, but also produce diesel and gasoline with high octane number. The core technology of TMP is the stratified injection of light and heavy feedstock, prompt termination of reactions of butylenes and olefins in light gasoline by heavy oil (such as atmospheric residue and recycle oil) injection. The cracking of heavy oil continues, which guarantees production of propylene with high yield and selectivity, as well as light oil production.

Highly selective conversion of butylenes and olefins of light gasoline to propylene requires catalyst with high content of HZSM-5 zeolite, reactions occurring at high temperature and in ultra-short residence time (about 10⁻² s), and full contacting of highly-dispersed reactant in molecule form with catalyst.

High reaction temperature is easily achieved by contacting butylenes and olefins of light gasoline with catalysts from the regenerator. High fluidized catalyst density, which can be realized by adjusting gas flow rate in riser reactor, can meet the requirement of full contacting of light feedstock with catalyst. Nevertheless, it is difficult to achieve ultra-short reaction time industrially. Currently, the contacting time of butylenes and olefins of light gasoline with catalyst alone is controlled in 0.3˜0.5 s at the TMP industrial unit. There are two measures for further decreasing the reaction time; one is to reduce the height of the reactor for separate reaction of light feedstock, which brings too small space to arrange related apparatus and uneven catalyst fluidization; the other is to increase the gas linear velocity, which decreases the fluidization density and has negative influences on contacting of oil gas with catalyst and the reaction.

In view of this, the present invention is proposed.

### DESCRIPTION OF THE INVENTION

An objective of this invention is to provide a riser reactor for the purpose of propylene production; by combining with stratified injection of heavy oil and light olefins, the yield and selectivity of propylene was improved, accompanied with production of diesel and gasoline with high octane number.

Another objective of this invention is to provide a method for propylene production using the above-mentioned riser reactor, which decreases yield of dry gas, and improves yield and selectivity of propylene.

A riser reactor, proposed by this invention, comprises pipelines with an upper part and a lower part, the upper part is a straight pipeline, and the lower part is a expanding-diameter pipeline, and the expanding-diameter pipeline is a circular truncated cone-shaped pipeline with an angle of 1˜60° between generatrix and axes, wherein, the diameter of upper surface of the circular truncated cone-shaped pipeline is larger than or equal to the diameter of the straight pipeline.

Preferentially, the diameter of upper surface of the circular truncated cone is equal to the diameter of the straight pipeline.

Preferentially, the angle between generatrix and axes of the circular truncated cone is in the range of 5 to 30°.

The height of the circular truncated cone-shaped pipeline ranges between 10 and 5000 mm, preferentially 2000˜5000 mm.

The diameter of the straight pipeline varies with different processing capacity of the unit, provided that the gas linear velocity in axial direction is controlled in the range of 3 to 25 m/s, preferentially 5 to 20 m/s.

In the case of a fixed feedstock processing capacity and gas linear velocity in axial direction, the straight pipeline diameter of the riser reactor can be obtained by technical personnel of this field.

In the above-mentioned riser reactor, a ring-shaped feeding pipeline with multiple nozzles is located at the bottom of the expanding-diameter pipeline.

Multiple nozzles can be evenly or unevenly set on the ring-shaped feeding pipeline; preferentially, the nozzles are evenly set every 5 to 300 mm in this invention; more preferentially, the nozzles are evenly set every 20 to 300 mm.

The angle between the nozzle and the axes of the circular truncated cone is in the range of 1 to 60°, preferentially 5 to 30°.

Light olefins are injected into the reactor through the ring-shaped feeding pipeline.

Atomizing nozzles for heavy oil injection are set above the expanding-diameter pipeline of the riser reactor.

A riser reactor with structure of changing-diameter was referred in study on particle flow characteristics in new structure FCC riser (ref: Petroleum Refinery Engineering, 2007, Vol.: 37, Issue: 10), but there is a straight pipeline behind the expanding-diameter pipeline, and the height of the former is larger than that of the latter. High fluidized catalyst density is achieved by decreasing the gas flowing rate, which prolongs the contacting time of oil gas with catalyst and reduce selectivity and yield of propylene.

Different from riser reactor with changing diameter disclosed by the prior art, no straight pipeline exists in the expanding-diameter part of the riser reactor in this invention (the straight pipeline in the expanding-diameter part shown in the drawing is set only for installing the ring-shaped feeding pipeline). The high fluidized catalyst density can be realized by wall effect without decreasing the gas flowing rate. This structure enables achieving high fluidized catalyst density with decreased contacting time of oil gas with catalyst below 0.3 s, which improves yield and selectivity of propylene.

The light olefins mentioned in this invention mainly include the olefins with 4 to 6 carbon atoms.

The heavy oil is defined as the crude oil with density higher than 0.93 g/cm³ at 20° C., or the oil with atmospheric boiling point higher than 350° C., such as atmospheric residue, vacuum residue, vacuum gas oil, coked gas oil and hydrocracking tail oil.

This riser reactor can be applied in any reaction/regeneration system of FCC unit disclosed by the prior art.

The system usually includes a riser reactor, a disengager and a regenerator. The regenerated catalyst of high temperature from regenerator flows upward along the riser reactor under the action of pre-fluidizing and pre-lifting steam. The catalyst mixed with the atomized feed from the nozzles, leads to crack of the feed in the process of upward flowing.

After being separated by the cyclone separator at the top of the riser reactor, the oil gas leaves the reaction/regeneration system to enter into a fractionator, while the deactivated catalyst enters into the regenerator for regeneration by coke-burning with air oxidation.

To realize another objectivity of this invention, a method of propylene production is proposed. Light olefins and heavy oil employed as feedstock are injected into the riser reactor from the nozzles of the ring-shaped feeding pipeline and the atomizing nozzles respectively, for fluidized catalytic cracking reactions; the average gas velocity in axial direction in the expanding-diameter pipeline of the riser reactor is in the range of 0.1 to 5 m/s, the outlet temperature of the riser reactor is 460 to 600° C., and the light olefins are no more than 50% of the total feedstock by weight.

Preferentially, the outlet temperature of the riser reactor is 480˜550° C.

Preferentially, the content of the light olefins in the feedstock is 5˜30 wt %.

Stratified injection of light olefins and heavy oil are employed in this invention. Light olefins interact with the catalyst firstly without influencing the catalyst activity for little coke formation, which not only has no negative effect on heavy oil conversion, but also promotes the conversion by increasing the catalyst-to-oil ratio. The extent of improvement is dependent on the ratio between the two feeds, the higher amount of the light olefins recycling, the greater improvement of the catalyst-to-oil ratio; however, conversion of heavy oil decreases as a result of the decreased catalyst temperature if excessive recycling of light olefins is employed. Therefore, the ratio of light olefins to heavy oil feeding should be controlled below 50%, preferentially 5˜30%.

Preferentially, average gas velocity in axial direction of the expanding-diameter pipeline of the riser reactor is 0.2˜2 m/s.

Average gas linear velocity in axial direction of the straight pipeline of riser reactor is 3˜25 m/s, preferentially 5˜20 m/s.

Preferentially, pre-heating temperature of the light olefins is from room temperature to 200° C., and that of the heavy oil is 150˜330° C.

Preferentially, linear velocity at the outlet of the light olefins and heavy oil injection is no more than 30 m/s and 70 m/s, respectively.

The method for propylene production by this invention adopts the special structure of expanding-diameter pipeline of the riser reactor and the stratified injection of light olefins and heavy oil, which promotes the contacting efficiency of oil gas with catalyst, shortens the residence time of oil gas in the reactor and improves yield and selectivity of propylene product.

The injection of heavy oil above the expanding-diameter pipeline terminates the light olefins reaction through expelling them from active sites by heavy oil of great absorbability, and through the lowering temperature, which ensures propylene selectivity of the light olefin conversion. The content of long-chain molecules is high in heavy oil, and they tend to produce dry gas due to thermal reactions, which decreases total yield of the target products (total yield of the target products is defined as the sum of the yield of liquefied petroleum gas (including propylene), gasoline and diesel). The injection of heavy oil above the expanding-diameter pipeline leads to decreased occurrences of thermal reaction by avoiding direct contact of heavy oil with catalyst of high temperature, and thus reduces yield of dry gas.

The catalyst-to-oil ratio is determined by the heat balance in riser reactor of circulated fluidized bed. In conventional FCC process, the catalyst-to-oil ratio can only be improved by increasing the temperature of the riser outlet if the unit remains the same, and this measure results in higher yield of dry gas. In this invention, the catalyst temperature has already been substantially reduced for heavy oil injection due to the previous reaction of light olefins, in the case of stratified injection of heavy oil and light olefins. The catalyst-to-oil ratio can be significantly improved by stratified injection, compared with feeding heavy oil alone, under the condition that temperature of the riser outlet remains.

The catalyst employed by this invention is the conventional catalyst for heavy oil catalytic cracking in this field.

Compared with the riser reactor of the prior art, advantages of the reactor and method thereof for propylene production by stratified injection of heavy oil and light olefins in this invention mainly lies in:

The reactor with special structure for stratified injection can make full use of the positive sides of gas velocity, reactor structure and feeding mode, and create reaction environment of high fluidized catalyst density and ultra-short reaction time, which is beneficial for light olefins conversion to propylene with high yield and selectivity. The reactor is also favorable for heavy oil conversion with higher conversion level, higher propylene yield and selectivity, and lower dry gas yield by improving the catalyst-to-oil ratio but without increasing temperature of the riser outlet.

Propylene selectivity is improved by the stratified injection of heavy oil and light olefins. Dry gas yield is reduced, accompanied with higher propylene yield and production of diesel and gasoline of high octane number, by avoiding direct contact of heavy oil with catalyst of high temperature.

### BRIEF DESCRIPTION OF THE DRAWING

FIG. 1 Schematic diagram of the riser reactor proposed by this invention

### EMBODIMENTS

The preferred embodiments of this invention are further described by the specific examples combined with the accompanying drawing, but they should not be interpreted as limitation of the scope of this invention.

### Example 1

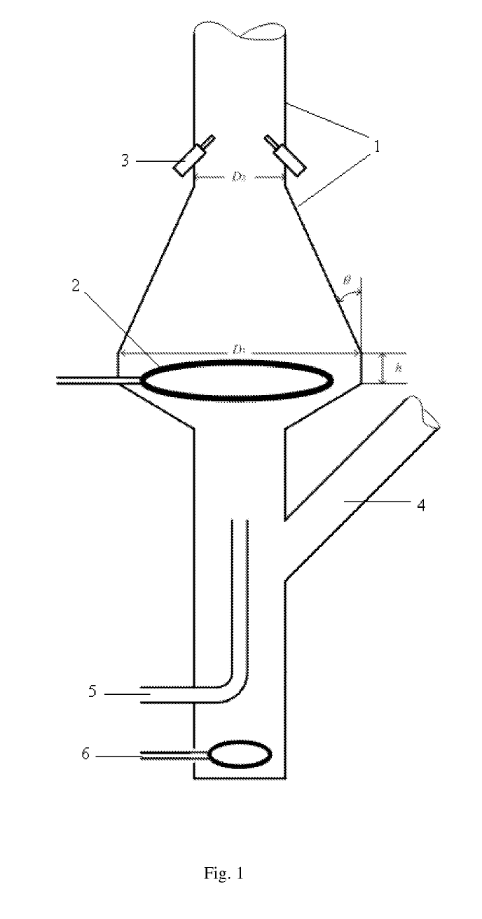

This invention provides a riser reactor of recycling fluidized bed unit. FIG. 1 shows the schematic diagram of the riser reactor proposed by this invention.

As shown in FIG. 1, the riser reactor 1 comprises pipelines with an upper part and a lower part, the upper part is a straight pipeline, and the lower part is an expanding-diameter pipeline in a circular truncated cone shape, with an angle of 1 to 60° between the generatrix and the axes, preferentially 5 to 30°. The height of the circular truncated cone-shaped pipeline is in the range of 10 to 5000 mm, preferentially 2000 to 5000 mm. The diameter of the straight pipeline is dependent on the processing capacity industrially, provided that the gas linear velocity in axial direction is controlled in the range of 3 to 25 m/s. In FIG. 1, diameter of the upper face of the circular truncated cone is larger than or equal to diameter of the straight pipeline, as long as the expanding-diameter structure exists in the riser reactor.

A ring-shaped feeding pipeline 2 with multiple are located at the bottom of the expanding-diameter pipeline and above the inclined pipeline 4 of the regenerator of the riser reactor 1. The nozzles (not shown in FIG. 1) are evenly set every 5 to 300 mm on the ring-shaped feeding pipeline 2; preferentially, the nozzles are evenly set every 20 to 300 mm. The angle between the nozzle and the axes of the circular truncated cone is in the range of 1 to 60°, preferentially 5 to 30°. A short straight pipeline with a height of h located around the ring-shaped feeding pipeline 2 is set for installation of the feeding pipeline, shown in FIG. 1. There are atomizing nozzles 3 for heavy oil injection located above the expanding-diameter part of the riser reactor.

### Example 2

In this example, propylene is produced by a circulating fluidized bed unit, using the riser reactor described in the above example 1.

Light olefins and heavy oil are injected into the riser reactor by the nozzles on the ring-shaped feeding pipeline and atomizing nozzles, respectively. The content of light olefins in the feedstock is 20 wt. %. The light olefins are pre-heated to 120° C. for injection, and the pre-heating temperature for heavy oil injection is 240° C. The average gas velocity in axial direction in the expanding-diameter part of the riser reactor is 1 m/s, and that in straight pipeline is 15 m/s. The outlet temperature of the riser reactor is 520° C. In the reactor-regenerator system of FCC unit, the regenerated catalyst of high temperature from the regenerator flows upward along the riser reactor under the action of pre-fluidizing and pre-lifting steam. It mixes with the atomized feeds from the nozzles, leading to crack of the feed in the process of upward flowing and the products including propylene.

### Example 3

In this example, propylene is produced by a circulating fluidized bed unit, using the riser reactor described in the above example 1.

Light olefins and heavy oil are injected into the riser reactor by the nozzles on the ring-shaped feeding pipeline and atomizing nozzles, respectively. The content of light olefins in the feedstock is 5 wt. %. The light olefins are pre-heated to 200° C. for injection, and the pre-heating temperature for heavy oil injection is 150° C. The average gas velocity in axial direction in the expanding-diameter part of the riser reactor is 0.2 m/s, and that in straight pipeline is 20 m/s. The outlet temperature of the riser reactor is 460° C. In the reactor-regenerator system of FCC unit, the regenerated catalyst of high temperature from the regenerator flows upward along the riser reactor under the action of pre-fluidizing and pre-lifting steam. It mixes with the atomized feed from the nozzles, leading to crack of the feed in the process of upward flowing and the products including propylene.

### Example 4

In this example, propylene is produced by a circulating fluidized bed unit, using the riser reactor described in the above example 1.

Light olefins and heavy oil are injected into the riser reactor by the nozzles on the ring-shaped feeding pipeline and atomizing nozzles, respectively. The content of light olefins in the feedstock is 50 wt. %. The light olefins are pre-heated to 80° C. for injection, and the pre-heating temperature for heavy oil injection is 300° C. The average gas velocity in axial direction in the expanding-diameter part of the riser reactor is 5 m/s, and that in straight pipeline is 10 m/s. The outlet temperature of the riser reactor is 600° C. In the reactor-regenerator system of FCC unit, the regenerated catalyst of high temperature from the regenerator flows upward along the riser reactor under the action of pre-fluidizing and pre-lifting steam. It mixes with the atomized feed from the nozzles, leading to crack of the feed in the process of upward flowing and the products including propylene.

### Example 5

In this example, propylene is produced by a circulating fluidized bed unit, using the riser reactor described in the above example 1.

Light olefins and heavy oil are injected into the riser reactor by the nozzles on the ring-shaped feeding pipeline and atomizing nozzles respectively. The content of light olefins in the feedstock is 30 wt. %. The light olefins are injected at room temperature, and the pre-heating temperature for heavy oil injection is 240° C. The average gas velocity in axial direction in the expanding-diameter part of the riser reactor is 2.0 m/s, and that in straight pipeline is 5 m/s. The outlet temperature of the riser reactor is 520° C. In the reactor-regenerator system of FCC unit, the regenerated catalyst of high temperature from the regenerator flows upward along the riser reactor under the action of pre-fluidizing and pre-lifting steam. It mixes with the atomized feed from the nozzles, leading to crack of the feed in the process of upward flowing and the products including propylene.

### Example 1 of Experiment

The results of propylene production by circulating fluidized bed units with two different structure of expanding-diameter part of riser reactor are compared in this example, and stratified injection is adopted in both units.

The riser reactor as a reference (straight pipeline used as expanding-diameter pipeline): the expanding-diameter pipeline at the bottom of the riser reactor is a straight pipeline, with an internal diameter of 62 mm and a height of 500 mm. Internal diameter of the other part of the riser is 10 mm, and total height of the riser is 9000 mm. The ring-shaped pipeline with upward openings (every 5 mm) and external diameter of 4 mm is installed at the bottom of expanding-diameter part of the riser reactor for light olefins injection. Atomizing nozzles for heavy oil injection are set at the bottom of non-expanding-diameter part of the riser reactor.

The riser reactor provided by this invention: expanding-diameter pipeline at the bottom of the riser reactor is in a shape of circular truncated cone, with a height of 300 mm and an angle of 5° between the generatrix and the axes. A straight pipeline with a height of 200 mm is set for installing the ring-shaped pipeline, and it located at the bottom of the expanding-diameter pipeline. The internal diameter of other parts of the riser reactor, except the expanding-diameter part, is 10 mm, and the total height of the riser is 9000 mm. The angle between the nozzle on the ring-shaped pipeline and axes of the circular truncated cone is 5°. Other setups are the same as referent riser reactor described above.

The feedstock and operating parameters (including feeding amount, feeding rate, and temperatures, etc) are kept the same in the two cases.

The properties of feedstock are shown in Table 1. The light olefins are a mixture of butylenes and butane with the content of butylenes of 80%, and light gasoline with the content of pentene and hexane added up to 65%.

Operating parameter: Feeding rate of heavy oil, C₄ mixture and light gasoline is 2, 0.3 and 0.4 kg/h, respectively. The reaction temperature which refers to temperature of the riser outlet is 520° C.; the catalyst-to-oil ratio is 10 for heavy oil; the average residence time of the oil gas in riser reactor is 1.6 s.

Table 2 shows the results of product distribution of catalytic cracking experiments on the two different units.

TABLE 1
Properties of feedstock

| Items | | | Unit | Heavy oil |
|---|---|---|---|---|
| Density (20° C.) | | | kg/m³ | 898.8 |
| | Viscosity | 80° C. | mm²/s | 4.47 |
| | | 100° C. | mm²/s | 3.16 |

| | | |
|---|---|---|
| Condensation point | ° C. | 24 |
| Carbon residue of electric furnace | wt % | 0.147 |
| Average molecular weight | g/mol | 413.7 |
| SARA composition | Saturated | 58.36 |
| wt % | Aromatics | 25.15 |
| | Resin | 16.3 |
| | Asphaltene | 0.19 |
| Element composition | C | 86.92 |
| wt % | H | 11.57 |
| | S | 0.62 |
| | N | 0.45 |
| Heavy metal | Ni | 0.081 |
| μg/g | V | 0.02 |
| | Fe | 162 |
| | Ca | 0.98 |
| Simulated distillation | IBP | 257 |
| ° C. | 10% | 353 |
| | 30% | 377 |
| | 50% | 391 |
| | 70% | 400 |
| | 90% | 416 |
| | FBP | 446 |

TABLE 2
Products distribution

| | | Expanding section in | Expanding section in |
|---|---|---|---|
| | | the form of a straight | the form of a reversed |
| Product distribution | | pipeline | “V”-shaped |
| Yield | Dry gas | 9.99 | 5.35 |
| wt. % | LPG | 34.82 | 35.13 |
| | Gasoline | 18.77 | 23.42 |
| | Diesel | 17.77 | 19.23 |
| | Heavy oil | 8.18 | 6.33 |
| | Coke | 10.47 | 10.54 |
| | Propylene | 22.29 | 24.7 |

The employment of the expanding-diameter pipeline in a circular truncated cone shape (or considered as a reversed “V”-shaped) can provide recycling C₄ mixture and olefins of light gasoline with the reaction environment of high fluidized catalyst density and short reaction time. Comparing with the riser reactor with expanding section in straight line, the reaction time is shortened, and the occurrences of backmixing are reduced. As a result, the reaction selectivity is improved; the yield of dry gas decreases substantially; the yield and selectivity of propylene increase evidently; and the conversion of heavy oil improves in a small extent.

Thus it can be seen that the new riser reactor with a reversed “V”-shaped expanding-diameter section and stratified injection, is effective and advantageous in promoting selective conversion of light olefins, increasing selectivity and yield of propylene, improving heavy oil conversion.

Similar results are obtained on the unit described in example 1 employing operation parameters described in examples 2˜5 by the first inventor. The yield of dry gas decreases substantially, the yield and selectivity of propylene increases evidently, and the conversion of heavy oil also improves in a small extent.

1. A riser reactor, comprising pipelines with an upper part and a lower part, the upper part being a straight line, and the lower part being a expanding-diameter pipeline, and the expanding-diameter pipeline being a circular truncated cone-shaped pipeline with an angle ranged of 1 to 60° between generatrix and axes, wherein, the diameter of upper surface of the circular truncated cone being larger than or equal to the diameter of the straight pipeline.

2. A riser reactor according to claim 1, wherein the diameter of upper surface of the circular truncated cone-shaped pipeline is equal to the diameter of the straight pipeline.

3. A riser reactor according to claim 1, wherein the angle between generatrix and axes of the circular truncated cone is in the range of 5 to 30°.

4. A riser reactor according to claim 1, wherein the height of the circular truncated cone-shaped pipeline ranges between 10 to 5000 mm.

5. A riser reactor according to claim 4, wherein the height of the circular truncated cone-shaped pipeline ranges between 2000 to 3000 mm.

6. A riser reactor according to claim 1, wherein a ring-shaped feeding pipeline with multiple nozzles is located at the bottom of the expanding-diameter pipeline.

7. A riser reactor according to claim 6, wherein the nozzles on the ring-shaped feeding pipeline are set every 5 to 300 mm.

8. A riser reactor according to claim 7, wherein the nozzles on the ring-shaped feeding pipeline are set every 20 to 300 mm.

9. A riser reactor according to claim 7, wherein an angle between the nozzle and the axes of the circular truncated cone-shaped pipeline is in range of 1 to 60°.

10. A riser reactor according to claim 9, wherein the angle between the nozzle and the axes of the circular truncated cone-shaped pipeline is in range of 5 to 30°.

11. A method for propylene production by the riser reactor of claim 1 comprising:
light olefins and heavy oil employed as feedstock being injected into the riser reactor from the nozzles of the ring-shaped pipeline and the atomizing nozzles, respectively, for fluidized catalytic cracking reactions; the average gas velocity in axial direction in the expanding-diameter part of the riser reactor being the range of 0.1 to 5 m/s, the outlet temperature of the riser reactor being 460 to 600° C., and the light olefins being no more than 50% of the total feedstock by weight.

12. A method according to claim 11, wherein the content of the light olefins in the feedstock is 5 to 30 wt %.

13. A method according to the claim 11, wherein an average gas linear velocity in axial direction of the straight pipeline of the riser reactor is 3 to 25 m/s.

14. A method according to the claim 13, wherein the average gas linear velocity in axial direction of the straight pipeline of the riser reactor is 5 to 20 m/s.

15. A method according to the claim 11, wherein an average gas velocity in axial direction of the expanding-diameter pipeline of the riser reactor is 0.2 to 2 m/s.
